(19) United States
(12) Patent Application Publication
(10) Pub. No.: US 2002/0049489 A1
(43) Pub. Date: Apr. 25, 2002

(54) Prosthesis and method of making a prosthesis having an external support structure

(76) Inventors: Steve A. Herweck, Nashua, NH (US); Paul Martakos, Pelham, NH (US); Chad Carlton, Nashua, NH (US)

Correspondence Address: LAHIVE & COCKFIELD, 28 STATE STREET, BOSTON, MA, 02109, US

(21) Appl. No.: 09/903,374

(22) Filed: Jul. 11, 2001

Related U.S. Application Data
(60) Provisional application No. 60/217,884, filed on Jul. 11, 2000.

(51) Int. Cl. A61F 2/06

(52) U.S. Cl. 623/1.13; 623/901; 623/1.33

(57) ABSTRACT

A prosthesis and a method of making a prosthesis having a needle containment and support structure that minimizes needle point plowing and/or needle scoring and inhibits delamination of the support structure during cannulization of the prosthesis. The prosthesis includes a first tube of expanded polytetrafluoroethylene (ePTFE), a polymer membrane, preferably ePTFE, positioned about the exterior surface of the first tube, and at least one support structure wound along a winding axis about the membrane to form axially spaced-apart ridges on the membrane. The support structure is a bead, filament, or similar structure that is wound about the exterior surface in a helical or spiral pattern to form the spaced apart-ridges. Alternatively, a plurality of spaced support rings can be employed to form the ridges. The ridges are preferably spaced apart a distance effective to direct a needle to a puncture site at an angle that inhibits needle plowing and hole enlarging, approximately less than 1.5 times the outer diameter of the needle. The polymer membrane has a microstructure selected to facilitate bonding of the support structure to the membrane and inhibit delamination of the support structure. In particular, substantially all the nodes forming the microstructure of the membrane are oriented at angle other 0° relative to the winding axis of the support structure. Preferably, substantially all the nodes forming the microstructure of the membrane are oriented in a direction substantially perpendicular to the winding axis of the support structure.

### BACKGROUND OF THE INVENTION

Synthetic vascular prostheses are frequently used to replace native blood vessels to provide dialysis needle access or cannulation. A common drawback to conventional synthetic vascular prostheses is the susceptibility of the prostheses to longitudinal tearing by the needle, commonly referred to as “needle point scoring” and/or “needle plowing”, which results in a needle hole larger than the outside diameter of the needle. An elongated needle hole is less likely to close and seal and can weaken the structural integrity of the wall of the prosthesis. Repeated cannulization can result in the formation of an aneurysm in the wall of the prosthesis, causing the prosthesis to fail. Needle point scoring and/or plowing shortens the effective life of the prosthesis and requires the prosthesis to be prematurely removed and replaced.

The penetrating angle of the needle can influence the amount of needle scoring or plowing. Lower penetration angles result in increased tearing of the prosthesis, while higher penetration angles can reduce the amount of scoring or plowing of the prosthesis wall. The penetration angle of the needle is difficult to control and is highly dependent on the skills and ability of the medical practitioner.

In an effort to better control the penetration angle of the needle and, thus, reduce needle plowing and scoring, it has been proposed to add a support structure to the exterior surface of the prosthesis in the form of a helically wound bead or a series spaced-apart rings. The support structure can be positioned to direct the needle at a higher penetration angle and can inhibit the needle from sliding longitudinally during cannulization. Prostheses employing such support structures are described in commonly owned U.S. Pat. No. 5,897,587, incorporated herein by reference. However, the support structure can frequently delaminate from the underlying prosthesis during cannulization due to insufficient bonding of the support structure to the prosthesis.

### SUMMARY OF THE INVENTION

The present invention overcomes the problems associated with conventional implantable prostheses by providing a prosthesis having a needle containment and support structure that minimizes needle point plowing and/or needle scoring and inhibits delamination of the support structure during cannulization of the prosthesis. The prosthesis can include an interface layer in the form of a membrane of polymer material between the exterior surface of the prosthesis and the support structure. The polymer membrane has a microstructure of nodes interconnected by fibrils effective to facilitate bonding of the support structure to the polymer membrane and inhibit delamination of the support structure from the membrane when the prosthesis is subject to cannulization.

In accordance with one aspect of the present invention, the prosthesis comprises a first tube of biologically compatible material, a polymer membrane positioned about the exterior surface of the first tube, and at least one support structure wound along a winding axis about the membrane to form axially spaced-apart ridges on the membrane. The polymer membrane can be a second tube positioned over the first tube or can be a wrap of polymer material applied to the exterior surface of the first tube. The polymer material of the membrane can be any polymer material suitable for use in an implantable prosthesis. The preferred polymer material is expanded polytetrafluoroethylene (ePTFE).

The polymer membrane can completely cover the longitudinal extent of the underlying first tube or can be applied to select points along the length of the first tube between the first tube and the support structure. In one preferred embodiment, the membrane can be wound about the first tube in a helical pattern.

The support structure can be a bead, filament, or similar structure that is wound about the exterior surface in a helical or spiral pattern to form the spaced apart-ridges. Alternatively, a plurality of spaced support rings can be employed to form the ridges. The ridges are preferably spaced apart a distance effective to direct a needle to a puncture site at an angle that inhibits needle plowing and hole enlarging. Applicants have determined that the spaced apart distance effective to inhibit needle plowing is a distance approximately less than 1.5 times the outer diameter of the needle. The support structure is preferably formed from a suitable polymer material and can include additional reinforcement such as an integral metal wire. In one preferred embodiment, the support structure is a bead having a diameter less than approximately 1 millimeter. The bead is preferably a bead of polytetrafluoroethylene (PTFE).

The polymer membrane or interface layer has a microstructure selected to facilitate bonding of the support structure to the membrane and to inhibit delamination of the support structure. In particular, substantially all the nodes forming the microstructure of the membrane are oriented at angle relative to the winding axis of the support structure. Preferably, the angle of the nodes is other than 0° relative to the winding axis of the support structure. In one preferred embodiment, substantially all the nodes forming the microstructure of the membrane are oriented in a direction substantially perpendicular to the winding axis of the support structure.

In accordance with a further aspect of the present invention, the first tube is constructed from a polymer material having a microstructure of nodes interconnected by fibrils, such as ePTFE. Preferably, the nodes forming the membrane are smaller than the nodes forming the first tube. Applicants determined that the smaller nodes forming the membrane improve bonding of the support structure to the membrane. In one preferred embodiment, the nodes forming the membrane are at least 10% smaller than the nodes forming the first tube.

In an additional aspect of the present invention, an outer polymer membrane can be placed over the support structure, the membrane, and the first tube. The outer polymer membrane is preferably bonded to the membrane and encloses the ridges.

The present invention also provides a method of making a prosthesis. The method comprises steps of providing a first tube of biologically compatible material, positioning a membrane of polymer material about the exterior surface of the first tube, and winding at least one support structure along a winding axis about the membrane to form axially spaced-apart ridges on the exterior surface. The membrane can have a microstructure of nodes interconnected by fibrils effective to facilitate bonding of the support structure to the membrane and inhibit delamination of the support structure from the membrane when the prosthesis is subject to cannulization.

In one aspect of the invention, the step of winding includes spacing the ridges apart a distance effective to direct a needle to a puncture site at an angle that inhibits needle plowing and hole enlarging. The spaced-apart distance is preferably less than 1.5 times the outer diameter of the needle.

In accordance with a further aspect of the present invention, the step of positioning the membrane includes wrapping the membrane about the first tube at selected points along the length of the first tube between the first tube and the support structure.

### BRIEF DESCRIPTION OF THE DRAWINGS

These and other features and advantages of the present invention will be more fully understood by reference to the following detailed description in conjunction with the attached drawings in which like reference numerals refer to like elements through the different views. The drawings illustrate principles of the invention and, although not to scale, show relative dimensions.

FIG. 1 is side elevational view of a prosthesis according to the teachings of the present invention;

FIG. 2 is a cross-sectional view of the prosthesis of FIG. 1 along the line 2-2;

FIG. 3 is a side elevational view in cross-section of the prosthesis of FIG. 1, illustrating the prosthesis being punctured with a needle;

FIG. 4 is a side elevational view of a prosthesis of the present invention, illustrating the microstructure of the polymer membrane and the underlying polymer tube;

FIG. 5 is a side elevational view of an alternative embodiment of the prosthesis of the present invention, illustrating the microstructure of the polymer membrane and the underlying polymer tube; and

FIG. 6 is a flow chart illustrating a method of making a prosthesis according to the teachings of the present invention.

### DETAILED DESCRIPTION OF THE PREFERRED EMBODIMENTS

The present invention provides prostheses and a method of making prostheses particularly suitable for implantation to replace a segment of a blood vessel and provide access for a dialysis needle or for cannulation. The prostheses of the present invention include an external support structure designed to direct and control needle penetration so as to minimize longitudinal tearing of the graft, commonly referred to as needle point scoring and/or plowing. In addition, the prostheses of the present invention provide a structure that improves bonding of the support structure to the exterior of the prosthesis and inhibits delamination of the support structure during cannulation.

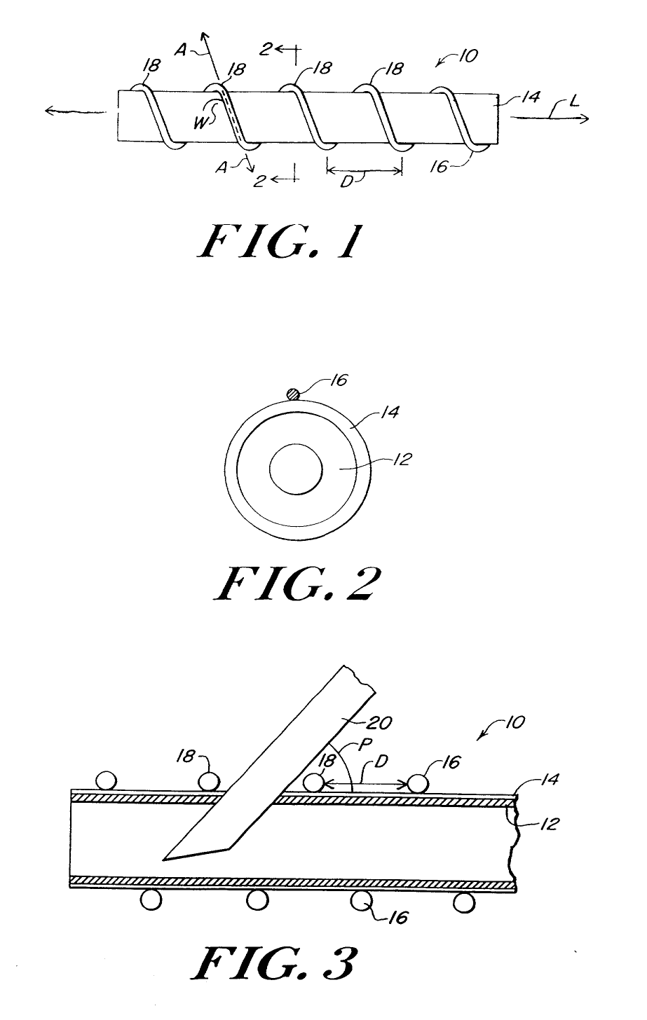

FIGS. 1-3 illustrate an embodiment of a prosthesis 10 of the present invention including a first tube 12, a membrane 14 positioned over the tube 12, and a support structure 16 helically wound about the membrane 14. The tube 12 can be constructed from biologically compatible materials suitable for implantation in vivo. In one preferred embodiment, the tube 12 is a tube of expanded polytetrafluoroethylene (ePTFE), such as an ePTFE graft available from Atrium Medical Corporation of Hudson, NH. The first tube 12, as well as the prosthesis 10, is not limited to the illustrated circular cross-section; other cross sections, such as rectangular, oval, elliptical, or polygonal, can be utilized.

Continuing to refer to FIGS. 1-3, the support structure 16 is wound about the membrane 14 in a spiral or helical pattern to form a series of discrete, spaced-apart ridges 18 along the exterior of the prosthesis 10. The support structure 16 can be a bead of polymer or other biocompatible material. In one preferred embodiment, the support structure 16 is a bead of solid, non-porous, and unexpanded polytetrafluoroethylene (PTFE). The support structure 16 can incorporate an additional reinforcement, such as a metal wire or the like embedded within the support structure 16. The diameter of the support structure is preferably less than or equal to 1 mm.

In an alternative embodiment, not shown, the support structure 16 can comprise a plurality, i.e., two or more, spaced-apart rings. The rings can be generally circular in shape, although are not limited to a circular configuration.

The support structure 16 is wound along a winding axis A about the membrane 14 at a winding angle W relative to the longitudinal axis L of the prosthesis 10. The winding angle W is preferably greater than 0° and can be up to 90°, as is the case when the support structure 16 is a ring or plurality of rings. The support structure 16 is preferably wound at a winding angle or pitch W such that the ridges 18 are spaced apart a distance, indicated by arrow D in FIGS. 1 and 3, effective to direct a needle to a puncture site at an angle that inhibits needle plowing and hole enlarging. Applicants determined that a spaced-apart distance D of approximately less or equal to than 1.5 times the outer diameter of the needle is effective to direct the needle between the ridges 18 at angle that inhibits needle plowing and hole enlarging.

Referring to FIG. 3, the support structure 16 operates as a needle hole containment system by guiding the needle 20 to the puncture site on the prosthesis at a higher penetration angle P, preferably greater than or equal to 30°. The most preferred penetration angle of the needle 20 is approximately 45°. The ridges 18 formed by the support structure 16 force the needle to drive in into the prosthesis at a predictable and desirable angle, independent of the skills of the medical practitioner forming the procedure. In addition, the support structure 16, and in particular the ridges 18 formed thereby, minimize the longitudinal motion of the needle 20 during needle puncturing. In this manner, the support structure 16 inhibits the needle 20 from making a hole significantly greater than the needle diameter and, thus, reduces the risk of aneurysm formation due to needle hole enlargement.

Moreover, applicants determined that the by closely spacing the ridges 18 in the manner described herein results in the formation of a smaller flat, slit shaped hole, that allows the prosthesis material to rapidly and effectively close or seal the hole upon removal of the needle. In contrast, conventional needle holes are generally cylindrical in shape or shaped like an elongated “V” and can resist closure and sealing of the needle hole. The slit-shaped hole can close and seal in the manner of a slit valve, allowing the prosthesis to achieve rapid hemostasis with minimal bleeding after needle withdrawal.

The membrane 14 is preferably constructed from a polymer material having a microstructure of nodes interconnected by fibrils, such as, for example, ePTFE. The microstructure of the membrane 14 is preferably structured to improve bonding of the support structure 16 to the membrane 14 and inhibit delamination of the support structure 16 during needle penetration and/or cannulation. In particular, substantially all the nodes forming the microstructure of the membrane 14 are oriented at angle other than 0° relative to the winding axis A of the support structure 16.

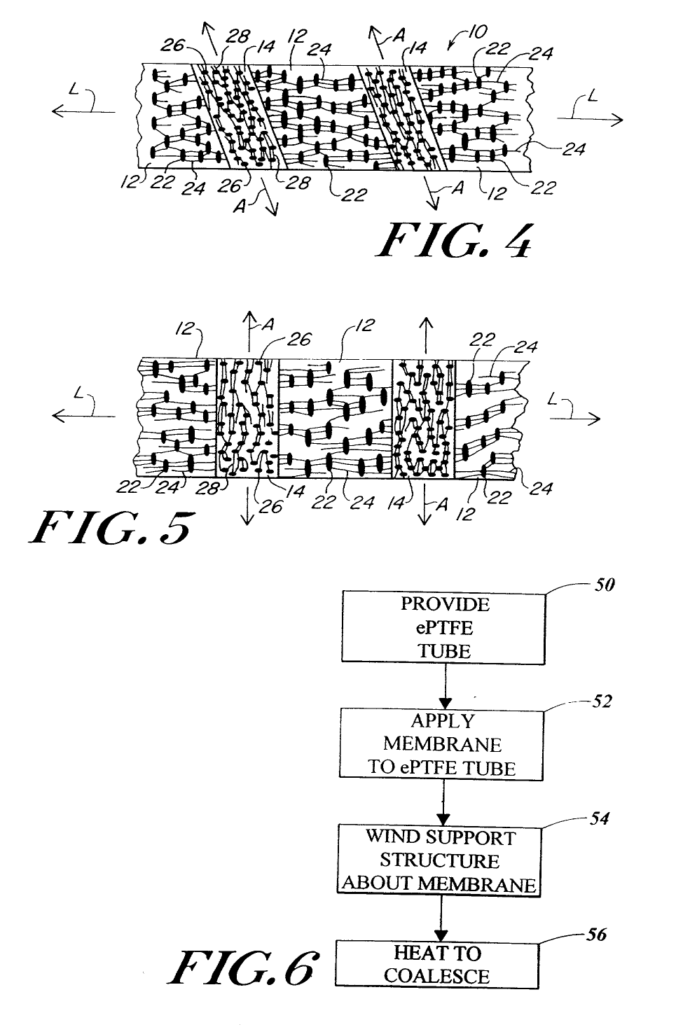

Referring to FIG. 4, the microstructure of the membrane 14 and the underlying tube 12 is illustrated prior to the application of the support structure 16. For purposes of this description, the microstructure of the membrane 14 and the tube 12 is exaggerated. The microstructure of the tube 12 is typical of a conventional ePTFE graft that has been formed through an extrusion and longitudinal expansion process. The nodes 22 of the graft microstructure are generally oriented perpendicular to the longitudinal axis L of the graft. The fibrils 24 interconnecting the nodes 22 are generally oriented in a direction parallel to the longitudinal axis L of the graft.

Continuing to refer to FIG. 4, the membrane 14 is a wrap of ePTFE applied at select locations along the length of the tube 12, preferably at locations upon which the support structure will be applied. In the preferred embodiment illustrated in FIG. 4, the membrane 14 is helically or spirally wrapped along the winding axis A of the support member 16. The nodes 26 forming the microstructure of the membrane 14 are generally oriented at an angle other than 0° relative to the winding axis A, i.e., in a direction other than parallel to the winding axis A. Applicants determined experimentally that this orientation of the nodes 26 forming the microstructure of the membrane results in improved bonding with the support structure while concomitantly minimizing delamination of the support structure 16 during needle puncture. The fibrils 28 of the membrane microstructure are oriented generally perpendicular to the nodes 26. In the preferred embodiment illustrated in FIG. 4, the nodes 26 are oriented generally perpendicular to the winding axis A and the fibrils 28 are oriented generally parallel to the winding axis A.

Applicants further determined that providing a membrane microstructure having nodes smaller in size than the nodes forming the microstructure of the underlying graft 12 can improve bonding with the support structure 16. Preferably, the nodes 26 are at least approximately 10% smaller than the nodes 22 of the graft. By reducing the size of the nodes 26, mores nodes 26 can be provided within the membrane microstructure. The increased number of nodes 26 result in a greater amount of nodal surface area for bonding with the support structure 16. Applicants believe this results in improved bonding with the support structure 16.

The membrane 14 is not limited to the helical or spiral orientation illustrated in FIG. 4 and described above, but can be wrapped in any orientation provided the membrane 14 is interposed at least partially between the support structure 16 and the exterior surface of the tube 12. For example, the membrane 14 can be applied in a plurality of discrete, spaced-apart rings, as illustrated in FIG. 5. The ring-like arrangement of the membrane 14 is particularly suited for embodiments of the prosthesis of the present employing a plurality of ring-shaped support structures. The nodes 26 are preferably oriented generally perpendicular to the winding axis A, which in this embodiment is perpendicular to the longitudinal axis L of the of the prosthesis 10. Thus, the nodes 26 are oriented generally parallel to the longitudinal axis L of the prosthesis 10, while the fibrils 28 are generally oriented perpendicular to the longitudinal axis L.

In an alternative embodiment, the membrane 14 can cover the entire circumferential and longitudinal extent of the tube 12. The membrane 14 thus acts an exterior cover or coating applied over the exterior surface of the tube 12.

One skilled in the art will appreciate that the membrane 14 is not limited to application as a wrap, but instead can be a separate tube of polymer material having the desired node size and nodal orientation. For example, the membrane 14 can be a separately formed second ePTFE tube that is applied to the exterior surface of the tube 12.

Alternatively, the exterior surface of the tube 12 can be provided with a nodal/fibril microstructure in which the nodes are oriented at an angle other than 0° relative to the winding angle. Plasma deposition techniques, such as those described in commonly owned, copending U.S. patent application Ser. No. 09/400,813, filed Sep. 22, 1999, and incorporated herein by reference, can be employed to provide the desired nodal orientation and, thus, eliminate the need for the membrane 14.

An outer membrane or layer of polymer material can optionally be placed over the support structure 16, the membrane 14, and the tube 12 to enclose the support structure 16. The outer membrane is preferably constructed from ePTFE or other polymer material suitable for in vivo implantation and is preferably bonded to the membrane 14.

A preferred method of manufacturing a prosthesis according to the present invention is illustrated in the flowchart of FIG. 6. In the first step of the method, an ePTFE tube is provided, step 50. The ePTFE graft can be formed by a conventional extrusion and expansion process. Suitable extrusion and expansion processes are described in commonly owned U.S. Pat. No. 5,897,587 and U.S. Pat. No. 5,433,909, both of which are incorporated herein by reference. The ePTFE tube is preferably placed on a mandrel and a membrane of polymer material is then applied to the exterior surface of the ePTFE tube. As discussed above, the membrane can be a wrap of ePTFE that is helically or spirally wound about the exterior surface of the ePTFE tube. The membrane is preferably selected to have a nodal/fibril microstructure in which substantially all the nodes are oriented an angle other than 0° relative to the winding axis of the support structure to be applied to the membrane. A support structure is wound along the winding axis about the membrane of polymer material, and thus, the ePTFE tube, step 54. The support structure can be a bead of PTFE, as described above. The support structure is preferably wound in a helical or spiral fashion to form a plurality of axially spaced-apart ridges on the membrane. The ridges are preferably spaced-apart a distance approximately less than or equal to 1.5 times the outer diameter of a dilation needle to be used with the graft to inhibit needle plowing and hole enlarging during needle puncturing and/or cannulization.

The ePTFE tube, including the membrane and the support structure, is heated to coalesce or bond together the support structure, the membrane, and the ePTFE tube, step 56.

It will thus be seen that the invention efficiently attains the objects made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

Having described the invention, what is claimed as new and desired to be secured by Letters Patent is:

1. A prosthesis for surgical implantation to replace a segment of a blood vessel, the prosthesis comprising:
a first tube of biologically compatible material having an exterior surface,
a membrane of polymer material positioned about the exterior surface of the first tube, and
at least one support structure wound along a winding axis about the membrane to form axially spaced-apart ridges on the membrane that enable the material to substantially close a hole that is created when the material is punctured by a needle or cannula, the membrane having a microstructure of nodes interconnected by fibrils effective to facilitate bonding of the support structure to the membrane and inhibit delamination of the support structure from the membrane.

2. The prosthesis of claim 1, wherein the support structure includes a metal wire.

3. The prosthesis of claim 1 further comprising an outer polymer membrane placed over the support structure, the membrane, and the first tube, the outer polymer membrane bonding to the membrane and enclosing the ridges.

4. The prosthesis of claim 1, wherein the ridges are spaced apart a distance effective to direct a needle to a puncture site at an angle that inhibits needle plowing and hole enlarging, the spaced apart distance being approximately less than or equal to 1.5 times the outer diameter of the needle.

5. The prosthesis of claim 1, wherein the first tube, the membrane, and the support structure are coalesced by heat.

6. The prosthesis of claim 1, wherein substantially all the nodes forming the microstructure of the membrane are oriented at angle relative to the winding axis of the support structure, the angle being other than 0° relative to the winding axis.

7. The prosthesis of claim 1, wherein substantially all the nodes forming the microstructure of the membrane are oriented in a direction substantially perpendicular to the winding axis of the support structure.

8. The prosthesis of claim 1, wherein the first tube is constructed from a polymer material having a microstructure of nodes interconnected by fibrils, the nodes forming the membrane being smaller than the nodes forming the first tube.

9. The prosthesis of claim 8, wherein the nodes forming the membrane are at least 10% smaller than the nodes forming the first tube.

10. A prosthesis for surgical implantation to replace a segment of a blood vessel, the prosthesis comprising:
a first tube of biologically compatible material having an exterior surface,
a membrane of polymer material positioned about the exterior surface of the first tube, and
a plurality of spaced-apart rings placed about the membrane to form axially spaced-apart ridges on the membrane that enable the material to substantially close a hole that is created when the material is punctured by a needle or cannula, the membrane having a microstructure of nodes interconnected by fibrils effective to facilitate bonding of the rings to the membrane and inhibit delamination of the rings from the membrane.

11. A prosthesis comprising:
an inner tube of polymer material having an exterior surface,
a membrane of polymer material positioned about the exterior surface of the inner tube, and
at least one support structure wound along a winding axis about the membrane to form axially spaced-apart ridges on the membrane that enable the material to substantially close a hole that is created when the material is punctured by a needle or cannula, the membrane having a microstructure of nodes interconnected by fibrils, the nodes being oriented at angle relative to the winding axis effective to facilitate bonding of the support structure to the membrane.

12. A method of making a prosthesis, the method comprising:
providing a first tube of biologically compatible material having an exterior surface,
positioning a membrane of polymer material about the exterior surface of the first tube, and
winding at least one support structure along a winding axis about the membrane to form axially spaced-apart ridges on the exterior surface that enable the material to substantially close a hole that is created when the material is punctured by a needle or cannula and the ridges being apart a distance effective to direct a needle to a puncture site at an angle that inhibits needle plowing and hole enlarging, the spaced apart distance being less than 1.5 times the outer diameter of the needle, the membrane having a microstructure of nodes interconnected by fibrils effective to facilitate bonding of the support structure to the membrane and inhibit delamination of the support structure from the membrane.
